(19) United States
(12) Patent Application Publication
(10) Pub. No.: US 2016/0361000 A1
(43) Pub. Date: Dec. 15, 2016

(54) DRUG APPLICATION DEVICE, DETECTION DEVICE, DRUG APPLICATION METHOD, AND DETECTION METHOD

(71) Applicant: Seiko Epson Corporation, Tokyo (JP)

(72) Inventor: Takafumi NODA, Matsumoto (JP)

(21) Appl. No.: 14/925,036

(22) Filed: Oct. 28, 2015

(30) Foreign Application Priority Data
Jun. 10, 2015 (JP) 2015-117658
Jun. 10, 2015 (JP) 2015-117659

(51) Int. Cl. A61B 5/1455; A61B 5/00; A61M 35/00; A61B 5/145

(52) U.S. Cl. CPC A61B 5/1455; A61B 5/14546; A61B 5/6833; A61M 35/00; A61B 5/445

(57) ABSTRACT

A drug application device irradiates terahertz light onto skin, outputs information concerning detection target cytokine determined from reflected rays of the terahertz light, and supplies a drug on the basis of the output information. A detection device irradiates terahertz light onto a patch attached to skin, and outputs information concerning detection target cytokine determined from reflected rays of the terahertz light.

### BACKGROUND

1. Technical Field

The present invention relates to a drug application device, a detection device, a drug application method, and a detection method.

2. Related Art

Allergic diseases such as atopic dermatitis have been treated by application of a steroid compound-containing drug or other such agents to the affected area (see, for example, JP-A-2003-313196). A patch test is also performed to specify causative sensitizing substances (allergens) in these diseases (see, for example, JP-A-6-238008).

The extent or conditions of inflammation are typically determined by visual inspection, and regimens such as applied drug quantities are determined on the basis of the observation result. However, as depicted in FIG. 5, the location 80 with externally observable allergic symptoms such as inflammation does not necessarily match the location 90 that has been sensitized to allergens. Specifically, a location sensitized to an allergen involves production of a certain cytokine (for example, thymus and activation-regulated chemokine, or TARC for short). However, locations with cytokines (locations sensitized to allergens) do not always show external abnormality such as inflammation in a diagnosis. Even in these locations where abnormality such as inflammation is not observed, a drug still needs to be applied as long as certain cytokines are produced. A problem arises when a drug is applied only in locations where external changes have occurred. In this case, the symptoms worsen in locations where the drug was not applied, and prolong the time to full recovery. When a drug is applied to both the location where external changes such as inflammation have occurred, and the location where such external changes are not observed, the drug would be applied in excess if the applied area includes a location where there is no production of certain cytokines. This may occur side effects or other problems.

In a patch test, a patch containing an allergen is attached to the skin, and removed after about 48 hours. The conditions of the skin where the patch was attached are then visually inspected for assessment.

However, it is not always easy to appropriately determine an allergic response with the method involving visual inspection. A common patch test with the long contact time of about 48 hours is also very stressful to subjects, requiring subjects, for example, not to remove the patch even when it is causing itching.

A common patch test also requires observing the skin conditions of the patch location after removing the patch. In the event where a clear allergic response was not recognized in the observation after the removal of the patch, the test needs to be repeated over a longer time with a new patch.

### SUMMARY

An advantage of some aspects of the invention is to provide a drug application device and a drug application method with which a drug can be desirably applied in appropriate quantities to appropriate skin locations where an allergic disease has occurred, and a detection device and a detection method that enable desirably evaluating an allergic response in skin.

The advantage can be achieved by the invention described below.

A drug application device according to an aspect of the invention includes: a photoirradiation unit that irradiates terahertz light onto skin; a light receiving unit that receives reflected rays of the terahertz light; a detector that outputs information concerning cytokine detected from the reflected rays; a drug supplying unit that supplies a drug to the skin; and a determination unit that determines a supply of the drug on the basis of the output information from the detector.

The drug application device can desirably apply an appropriate quantity of drug to an appropriate location of the skin affected by allergic disease.

It is preferable that the drug application device of the aspect of the invention includes a display unit that displays the information concerning cytokine.

With this configuration, for example, a user can easily and accurately grasp information concerning the cytokine contained in a predetermined region of the skin. The safety of the drug application device also improves.

It is preferable that the drug application device of the aspect of the invention includes a determination unit that determines a supply of the drug on the basis of whether the output value from the detector exceeds the threshold value.

With this configuration, for example, an appropriate drug can be more reliably applied in an appropriate amount to the required location of the skin (tested location). It also becomes possible to effectively prevent problems due to deficient or excessive quantities of drug being applied to locations of the skin (tested locations) as might occur when a user does not follow the correct procedures.

In the drug application device of the aspect of the invention, it is preferable that the drug supplying unit adjusts a supplied drug quantity according to the information from the detector.

With this configuration, an appropriate drug can be more reliably applied in an appropriate amount to the required location of the skin (tested location).

It is preferable that the drug application device of the aspect of the invention includes an application roller that rolls on the skin and applies the drug to the skin.

With the application roller, the drug can be desirably spread over a predetermined range of skin, and can be effectively prevented from being unevenly applied to the required application region by mistake. The drug application procedure also can become easier and smoother.

It is preferable that the drug application device of the aspect of the invention includes an extrusion roller that presses a container storing the drug and extrudes the drug out of the container.

With this configuration, the quantity of the supplied drug from the container can be more desirably adjusted, and an appropriate drug can be more reliably applied in an appropriate amount to the required location of the skin (tested location).

It is preferable that the drug application device of the aspect of the invention includes a drug container holder that holds a drug container containing the drug.

This allows the drug application device to stably hold the drug container, and further improves the operability (ease of operation) of the drug application device.

In the drug application device of the aspect of the invention, it is preferable that the detector outputs information concerning a content of TARC (thymus and activation-regulated chemokine).

With this configuration, the extent of allergic symptoms can be more appropriately determined, and it becomes possible to, for example, more desirably decide the type and the quantity of the drug to be applied.

A detection device according to another aspect of the invention includes: a photoirradiation unit that irradiates terahertz light; a light receiving unit that receives reflected rays of the terahertz light; a detector that outputs information concerning cytokine detected from the reflected rays; and a display unit that displays the information concerning cytokine.

The detection device can desirably obtain information concerning cytokine.

In the detection device of the aspect of the invention, it is preferable that the terahertz light irradiates a patch attached to skin.

With this configuration, an allergic response in skin can be desirably evaluated.

In the detection device of the aspect of the invention, it is preferable that the patch has a moisture content of 0.5 g/cm² or less per unit area in a region irradiated with the terahertz light.

With this configuration, cytokine can be detected with improved accuracy, and the allergic response in skin can be more desirably evaluated.

In the detection device of the aspect of the invention, it is preferable that the patch is configured from a material that contains at least one selected from the group consisting of polyethylene, polystyrene, polyvinyl chloride, and polyvinylidene chloride.

With this configuration, cytokine can be more desirably detected in the location where the patch is attached. It also becomes possible to effectively prevent problems such as entry of outside moisture, and efflux of allergens due to moisture so that the patch test can be desirably performed.

In the detection device of the aspect of the invention, it is preferable that the patch contains an allergen, and that an area of a part of the patch where the allergen is contained is 0.01 cm² to 0.25 cm².

With this configuration, the allergic response in skin can be accurately evaluated despite that the patch is smaller than patches used in common patch tests (patch tests involving evaluation by visual inspection). A burden on patients also can be reduced with the small patch (a patch with a small contact area between the skin and allergens) . The small patch also has other advantages, including shipping cost, storage space, and resource saving.

In the detection device of the aspect of the invention, it is preferable that the detector outputs information concerning a content of TARC (thymus and activation-regulated chemokine).

With this configuration, the extent of allergic symptoms can be more appropriately determined, and, for example, the type and the quantity of the drug to be imparted can be more desirably determined. It is also possible to, for example, desirably determine the allergen dose to be given to a patient in an oral challenge therapy.

In the detection device of the aspect of the invention, it is preferable that the terahertz light is produced with a photoconductive antenna by wavelength conversion from light emitted by a femtosecond laser light source.

A drug application method according to still another aspect of the invention includes: irradiating terahertz light onto skin; receiving reflected rays of the terahertz light; outputting information concerning detection target cytokine determined from the reflected rays; and supplying a drug to the skin on the basis of the information concerning cytokine.

The drug application method can desirably apply an appropriate quantity of drug to an appropriate location of the skin affected by allergic disease.

A detection method according to yet another aspect of the invention includes: irradiating terahertz light onto a patch attached to skin; receiving reflected rays of the terahertz light; and obtaining information concerning detection target cytokine determined from the reflected rays.

The detection method can desirably evaluate an allergic response in skin.

### BRIEF DESCRIPTION OF THE DRAWINGS

The invention will be described with reference to the accompanying drawings, wherein like numbers reference like elements.

FIG. 1 is a plan view schematically representing a preferred embodiment of a drug application device according to the invention.

FIG. 2 is a cross sectional view of the drug application device of FIG. 1, taken at a plane different from the cross section shown in FIG. 3.

FIG. 3 is a cross sectional view of the drug application device of FIG. 1, taken at a plane different from the cross section shown in FIG. 2.

FIG. 4 is a flowchart representing an example of a drug application method according to the invention.

FIG. 5 is a diagram explaining the positional relationship between a location where an allergic symptom such as inflammation is observed, and a location sensitized to an allergen.

FIG. 6 is a partial cross sectional view schematically representing a preferred embodiment of a detection device according to the invention.

FIG. 7 is a flowchart representing an example of a detection method according to the invention.

### DESCRIPTION OF EXEMPLARY EMBODIMENTS

Preferred embodiments are described below with reference to the accompanying drawings.

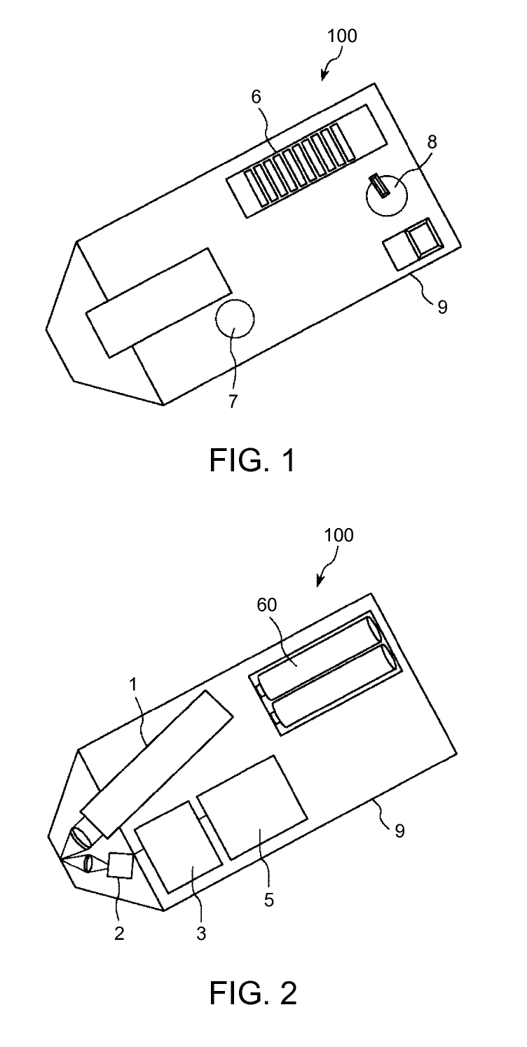

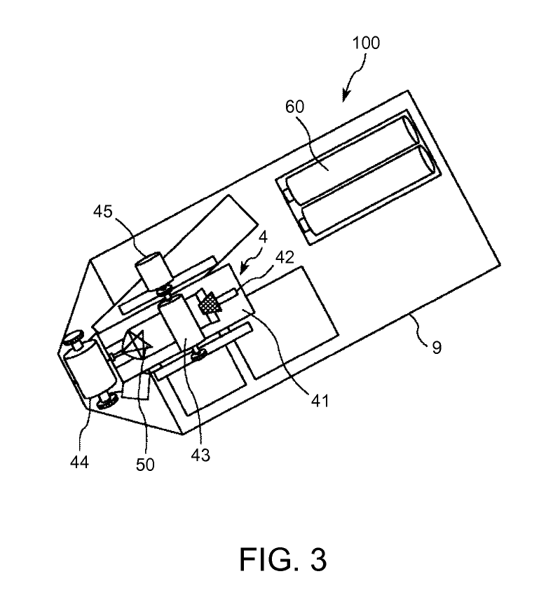

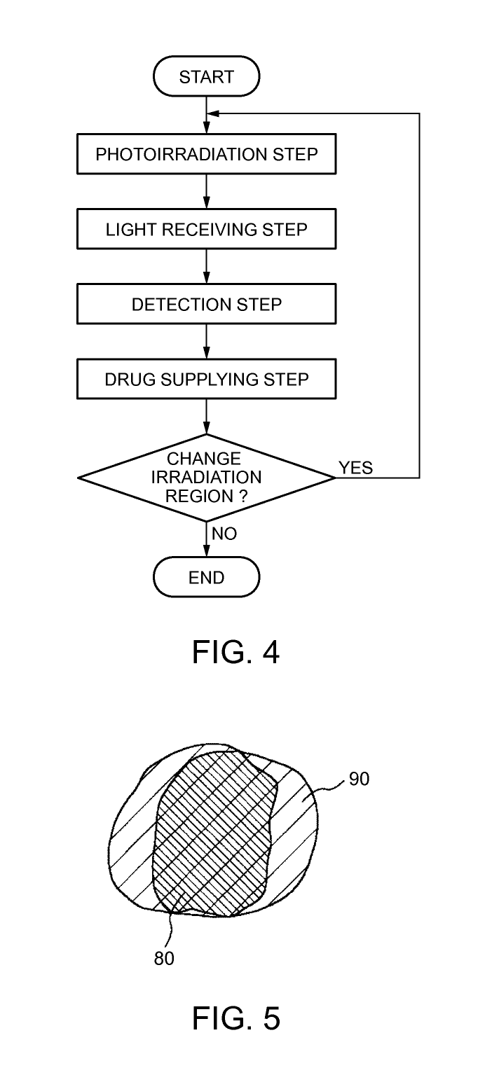

FIG. 1 is a plan view schematically representing a preferred embodiment of a drug application device (drug imparting device) according to the invention. FIG. 2 is a cross sectional view of the drug application device shown in FIG. 1 (taken on a plane different from the cross section shown in FIG. 3). FIG. 3 is a cross sectional view of the drug application device shown in FIG. 1 (taken on a plane different from the cross section shown in FIG. 2). FIG. 4 is a flowchart representing an example of a drug application method (drug imparting method) according to the invention.

As illustrated in FIGS. 1 to 3, a drug application device (drug imparting device) 100 includes a photoirradiation unit 1 that irradiates terahertz light onto skin, a light receiving unit 2 that receives reflected rays of the terahertz light, a detector 3 that outputs information concerning detection target cytokine determined from the reflected rays, and a drug supplying unit 4 that supplies a drug.

As represented in FIG. 4, the drug application method according to the invention includes a photoirradiation step of irradiating terahertz light onto skin, a light receiving step of receiving reflected rays of the terahertz light, a detection step of outputting information concerning detection target cytokine determined from the reflected rays, and a drug supplying step of supplying a drug to the skin as needed on the basis of the information concerning cytokine. The method optionally includes an irradiation region changing step of changing the terahertz light irradiation region. When changing the terahertz light irradiation region, the method performs the light receiving step, the detection step, and the drug supplying step for the new irradiation region.

As used herein, “terahertz light” is light that moderately passes through and reflects on skin, particularly the epidermis. As used herein, “cytokine” is a compound that absorbs terahertz light in a specific absorption spectrum that varies with the chemical structure.

The foregoing configuration thus enables desirably detecting cytokines produced in biological reactions concerning allergy in the skin (epidermis). This makes it possible to desirably apply (impart) a drug to the cytokine detected location.

The content of a predetermined cytokine in a location irradiated with the terahertz light can be determined by finding the intensity of the reflected rays of the terahertz light of a predetermined wavelength (the proportion relative to the intensity of the irradiated light). This desirably enables not only detection of the presence or absence of predetermined cytokine production but quantitative analysis of the cytokine. The detection result can thus be desirably used not only in deciding whether a drug needs to be applied (imparted) to the predetermined location but in determining the type or the quantity of the drug that needs to be applied (imparted). This can achieve an early remission of allergic disease, and effectively prevent the harmful effects of a prolonged treatment (e.g., side effects of drugs such as steroids), complications, and allergy march. A burden on patients also can be reduced because the extent of allergic symptoms can be evaluated in a noninvasive fashion without the need to collect samples. Further, with the quick acquisition of information concerning cytokine, a treatment such as drug application can be quickly and appropriately performed using the information, as soon as the information is obtained.

The attendance of a physician or other healthcare professional is also not necessary. The patient by himself or herself can operate the drug application device, and apply an appropriate quantity of a drug to an appropriate location.

Because the required drug quantity is determined not by the patient but by the device, a failure to apply the drug in the required quantity due to any anxiety the patient might have for the drug can be avoided to prevent worsening or protraction of the disease.

These desirable effects cannot be obtained with light other than terahertz light. For example, light having shorter wavelengths than terahertz light (for example, near infrared light) exposes the dermis through the epidermis, and the result is affected by dermis and blood vessels, which makes it difficult to accurately detect specific cytokines. With light having longer wavelengths than terahertz light (for example, milliwaves), the characteristic absorption spectrum of a cytokine will not be observed, and it is difficult to accurately detect specific cytokines.

The cytokine may be, for example, any of various interleukins (IL) such as IL-5, 6, 8, 13, and 33; TSLP (thymic stromal lymphopoietin); hematopoietic factors such as GM-CSF (granulocyte macrophage colony stimulating factor); and various chemokines, including CC chemokines such as CCL 2, 22 (MDC), and 24 (Extaxin-2), CXC chemokines such as CXCL 1 and 8, C chemokines, and CX3C chemokines.

Terahertz light has wavelengths of 0.5 THz to 10 THz. The photoirradiation unit 1 irradiates terahertz light in wavelengths of preferably 0.8 THz to 3.0 THz, more preferably 1.0 THz to 2.0 THz.

In this way, various cytokines can be quantitatively detected with improved accuracy.

The photoirradiation unit 1 may be installed in any position, as long as terahertz light can be irradiated (emitted). In the present embodiment, the photoirradiation unit 1 is a light source installed inside the casing (housing) 9 of the drug application device 100.

Examples of the photoirradiation unit (light source) 1 includes solid-state oscillators such as Gunn diodes, and resonant tunneling diodes; heat radiating light sources such as a mercury lamp, a blackbody furnace, and a halogen lamp; femtosecond light sources that generate a terahertz wave with a femtosecond laser; and optical electronics light sources using nonlinear optical crystals, photoconductive antennas, and the like.

Examples of the nonlinear optical crystals include DAST, BNA, GaSe, GaP, and OH1.

Examples of the photoconductive antennas include a dipole shaped photoconductive antenna (PCA).

The terahertz light irradiated by the photoirradiation unit (light source) 1 may be light with more than one frequency (wavelength) component.

For example, the terahertz light irradiated by the photoirradiation unit (light source) 1 may contain at least one reference frequency (reference wavelength) for obtaining information concerning individual differences (for example, a skin tone under normal conditions), or the skin condition at the time of the measurement (for example, the quantity of a component (for example, collagen) other than the cytokine being detected for changes over a time course), and at least one measurement frequency (measurement wavelength) for obtaining information of cytokine levels in the skin.

In this way, the effect of components other than the detection target cytokine can be more desirably excluded, and the predetermined cytokine can be detected with improved accuracy. This makes it possible to more desirably determine conditions such as the quantity of the drug to be applied.

When irradiating terahertz light of measurement frequency and terahertz light of reference frequency, these may be irradiated at the same time or at different timings.

The terahertz light emitted from the photoirradiation unit 1 irradiates the skin at the tested location.

The terahertz light emitted by the photoirradiation unit 1 may irradiate the skin through a bandpass filter. In the configuration depicted in the diagrams, the single light source (photoirradiation unit 1) is shown as emitting terahertz light onto the skin. However, the drug application device 100 may include a plurality of light sources (photoirradiation units 1).

The terahertz light that irradiates the skin at the tested location is reflected at the tested location (skin), and the reflected rays are received by the light receiving unit 2.

The light receiving unit 2 is configured from an optical sensor capable of detecting terahertz light.

The optical sensor used to form the light receiving unit 2 may be any of, for example, quantum sensors such as photodiodes, phototransistors, and photo ICs; thermal sensors such as thermocouples, pyroelectric sensors, and bolometers; and photoconductive antennas. The thermal sensors are advantageous in terms of miniaturizing the drug application device 100.

The light receiving unit 2 may include a single sensor, or may include a plurality of sensors that is arranged in lines on a chip or arrayed on a surface of a chip.

The light receiving unit 2 may be adapted to detect terahertz light of more than one frequency (wavelength).

For example, the light receiving unit 2 may be adapted to detect terahertz light of at least one reference frequency (reference wavelength) for obtaining information concerning individual differences (for example, a skin tone under normal conditions), or the skin condition at the time of the measurement (for example, the quantity of a component (for example, collagen) other than the cytokine being detected for changes over a time course), and terahertz light of at least one measurement frequency (measurement wavelength) for obtaining information of cytokine levels in the skin.

In this way, the effect of components other than the detection target cytokine can be more desirably excluded, and the predetermined cytokine can be detected with improved accuracy. This makes it possible to more desirably determine conditions such as the quantity of the drug to be applied.

The light receiving unit 2 may include a bandpass filter (for example, a filter that selectively passes light of a predetermined wavelength in the wavelength region of the terahertz light).

This further improves the detection accuracy of predetermined cytokine. The need for a frequency sweep also can be eliminated to simplify the device configuration.

Preferably, the bandpass filter selectively passes light of the wavelength corresponding to the absorption peak of the absorption spectrum of the terahertz light for a specific cytokine.

This further improves the detection accuracy of predetermined cytokine.

The accuracy of specifying different cytokines can improve when the bandpass filter is adapted to selectively pass light of different wavelengths corresponding to the characteristic absorption peaks (different absorption peaks) of different cytokines.

The detector 3 outputs information concerning the detection target cytokine determined from the reflected rays received by the light receiving unit 2.

Specifically, for example, the detector 3 obtain information concerning, for example, the presence or absence of a specific cytokine at the tested location, the quantity of the cytokine contained in the predetermined region, and the type of cytokine from the intensity of the reflected rays of a specific frequency (wavelength) received by the light receiving unit 2 (the intensity relative to the intensity emitted by the photoirradiation unit 1). The detector 3 then outputs the information.

Preferably, the detector 3 outputs information concerning a content of TARC (thymus and activation-regulated chemokine). The content information includes the presence or absence of TARC.

Compared to other cytokines, TARC is more prominently produced in locations sensitized to allergens in diseases such as atopic dermatitis. TARC also has an absorption spectrum that can be more easily distinguished from other substances with terahertz light. With the detector 3 outputting information concerning a TARC content, the extent of allergic symptoms can thus be more appropriately determined, and, for example, conditions such as the type and the quantity of the drug to be applied can be more desirably determined.

The detector 3 may be adapted to output a plurality of values based on measurements with a plurality of frequencies (wavelengths). More specifically, the detector 3 may be adapted to output information concerning the content of more than one cytokine determined from information concerning the reflection intensities of a plurality of wavelengths corresponding to the absorption wavelengths (absorption peaks) of more than one cytokine. This makes it possible to more desirably specify a disease or examine disease progression, and to more desirably select the type of the drug, or determine the quantity of the drug to be applied.

The detector 3 may be adapted to output a single value based on measurements with a plurality of frequencies (wavelengths). More specifically, the detector 3 may be adapted to output information concerning the content of a specific cytokine determined from information concerning the reflection intensities of a plurality of wavelengths (frequencies) corresponding to more than one absorption peak in the absorption spectrum of the cytokine. This makes it possible to improve the detection accuracy for a specific cytokine.

The drug application device 100 of the present embodiment includes a determination unit 5 that determines a supply of the drug on the basis of the output information from the detector 3.

In this way, for example, an appropriate drug can be more appropriately applied in an appropriate amount to the required location of the skin (tested location). With the determination unit 5 outputting the result to the drug supplying unit 4, it is possible to, for example, automatically apply a drug to the required location of the skin (tested location), and effectively prevent problems due to deficient or excessive quantities of drug being applied to locations of the skin (tested locations) as might occur when a user does not follow the correct procedures.

The determination unit 5 may be adapted to determine the need to apply (impart) a drug, for example, on the basis of whether the output value from the detector 3 exceeds the threshold value.

The threshold value may be appropriately varied for different drugs.

The determination unit 5 may be adapted to determine the applied drug quantity, for example, by comparing the output value from the detector 3 with values of a threshold table.

The threshold table may be varied for different drugs.

The determination unit 5 may be adapted to determine the applied drug quantity, for example, by substituting the output value from the detector 3 into an arithmetic expression.

The determination unit 5 may be adapted to determine the applied drug quantity, for example, by substituting the output value from the detector 3, and a drug-dependent numerical value into an arithmetic expression.

The determination unit 5 may be adapted to determine the applied drug quantity on the basis of more than one output value from the detector 3.

The drug supplying unit 4 supplies a drug to the skin containing cytokine.

The drug supplying unit 4 includes a tube holder (drug container holder) 41 that holds the tube (drug container) 50 storing a drug, a check unit 42 that checks the type of the drug stored in the tube (drug container) 50, an extrusion roller 43 that presses the drug tube 50 to extrude the drug contained in the drug tube 50, and an application roller 44.

The tube holder (drug container holder) 41 has a space for storing the tube (drug container) 50, and fixes a part of the tube (drug container) 50.

This allows the drug application device 100 to stably hold the tube (drug container) 50, and further improves the operability (ease of operation) of the drug application device 100.

The tube holder (drug container holder) 41 may be adapted to hold a commercially available drug product (a drug container storing a drug), or a drug container designed specifically for the drug application device 100.

The check unit 42 functions to check the type of the drug stored in the tube (drug container) 50 (the type of the drug container 50 containing a specific quantity of a specific drug).

The device is configured so that the drug is not applied when the drug stored in the tube (drug container) 50 installed in the tube holder (drug container holder) 41 is not the intended drug. This improves safety by effectively preventing the wrong drug from being inadvertently applied to skin.

The device is also configured so that, for example, the supplied drug quantity is adjusted, and the type of the cytokine to be detected is changed according to the type of drug. In this way, different drugs can be desirably used for different conditions, such as disease progression in a patient.

In the configuration shown in the diagram, the check unit 42 with the foregoing function checks the type of drug (type of the drug container 50 containing a specific amount of a specific drug) by the size of the tube (drug container) 50 itself, or by the position, the shape, and the size of the notched portion provided in the tube (drug container) 50.

Conditions such as the need to supply a drug, and the quantity of the drug to be supplied from the drug supplying unit 4 are decided, for example, on the basis of the output information from the determination unit 5. More specifically, the device is configured so that the desired quantity of drug is pushed out of the tube (drug container) 50 and supplied to the desired location of the tested location as the extrusion roller 43 applies a predetermined amount of pressure to the tube (drug container) 50 at a predetermined rate under the drive of a motor 45, using the output information from the determination unit 5.

In this way, the supplied drug quantity (extrusion amount) from the tube (drug container) 50 can be desirably adjusted, and an appropriate drug can be more reliably applied to the required location of the skin (tested location) in an appropriate amount.

The application roller 44 rolls on skin, and functions to apply the supplied drug from the drug container 50 to skin.

With the application roller 44, the drug can be desirably spread over a predetermined range of skin, and can be effectively prevented from being unevenly applied to the required application region by mistake. The drug application procedure also can become easier and smoother.

The drug application device 100 of the present embodiment includes a display unit 6 that displays information concerning cytokine based on the output information from the determination unit 5.

The display unit 6 allows users (e.g., patients, physicians, and nurses) to, for example, easily and accurately grasp information concerning the cytokine contained in the predetermined region of the skin. Users also can easily notice an error occurred in the drug application device 100, and the safety of the drug application device 100 improves. The display unit 6 also eliminates the need to connect the drug application device 100 to a display device or the like for the purpose of displaying information such as above. This allows users to recognize information concerning cytokine with a simple configuration.

The display unit 6 displays information concerning cytokine. Examples of the information concerning cytokine displayed in the display unit 6 include a cytokine content in a location irradiated with terahertz light, the need to apply a drug, and the required drug quantity for application.

With this information, users (e.g., patients, physicians, and nurses) can grasp appropriate information, and can effectively make use of the information for the treatment of a patient. Users also can desirably manage cytokine changes over a time course, or the past course of treatment, such as drug doses.

In the configuration shown in the diagram, the display unit 6 is a level meter. This helps intuitively grasp the relation between different values of cytokine. The level meter may be adapted to display levels in different colors. This makes it easier for users to grasp different levels.

The display unit 6 provided as a level meter in the configuration shown in the diagram may display information concerning cytokine in characters (e.g., numerical values) or images. For example, a distribution of cytokine contents at different locations of the region irradiated with terahertz light may be displayed in colors.

This makes it easier to visually determine the extent of allergic symptoms. It also becomes possible to desirably display a state of a relatively large region (for example, a region about 10 cm wide).

The display unit 6 also may be adapted to, for example, notify a level relative to the threshold value (for example, by turning on and off a light emitting device such as an LED).

The display unit 6 is not limited to visually displaying (notifying) information concerning cytokine, and may, for example, notify information by sound. For example, a buzzer may be used to notify a level relative to the threshold value. The sound may be languages, including, for example, Japanese, Chinese, English, French, and German.

When the display unit 6 is adapted to display (notify) information concerning cytokine by sound, the volume, the tone, and other characteristics of the sound may be varied for different values of cytokine.

The drug application device 100 of the present embodiment also includes an application switch 7 that allows a drug to be manually applied. The application switch 7 is operated (pressed in the configuration shown in the diagram) to supply a drug.

In this way, a user of the drug application device 100 can choose to apply (manually apply) a drug by operating the application switch 7, rather than using the result of the detection with terahertz light for application (automatic application).

It is also possible to apply a desired quantity of drug to the location where cytokine has been detected but the drug has not been applied, and to effectively prevent the drug from being applied in excess to the location where the drug has already been applied, for example, when irradiating terahertz light to a region that includes a location where the drug has been applied, and a location where the drug has not been applied. The drug can thus be more reliably applied in appropriate quantities in different locations of the region irradiated with terahertz light.

The applied drug quantity also can be desirably adjusted (corrected) from the displayed result in the display unit 6 according to factors such as individual differences (for example, disease progression, disease history, and body weight).

The drug application device 100 of the present embodiment also includes a select switch (application mode select switch) 8 used to select whether a drug should be automatically or manually applied.

With the select switch (application mode select switch) 8 set to automatic application mode, the drug is automatically applied using the terahertz light detection result. With the select switch (application mode select switch) 8 set to manual application mode, the drug application is allowed only through the operation of the application switch 7 (manual application), and any automatic drug application based on the result of terahertz light detection is suspended.

For example, the manual drug application (manual application mode) allows the device to be used solely for checking disease progression, without having to apply a drug. It is also possible to obtain both the effect of automatic drug application and the effect of manual drug application. More specifically, ease of drug application procedure can improve as a whole, enabling a drug to be applied in appropriate quantities in different locations, and the applied drug quantity to be desirably adjusted (corrected), for example, when irradiating terahertz light to a region that includes a location where the drug has been applied, and a location where the drug has not been applied.

The photoirradiation unit 1, the light receiving unit 2, the detector 3, the drug supplying unit 4, the determination unit 5, and other components of the device are housed in the casing (housing) 9, and are desirably protected.

The casing 9 also serves as a holder when operating the drug application device 100.

The foregoing procedures including irradiation of terahertz light are typically performed on at least one of the location sensitized to allergens, and the location that may have been sensitized to allergens. However, the procedures including irradiation of terahertz light may also be performed on a normal location that is not sensitized to allergens, in addition to at least one of the location sensitized to allergens, and the location that may have been sensitized to allergens.

In this way, by using the normal location as a reference location, the measurement result from the normal location (reference location) can be compared with the measurement results from the location sensitized to allergens, and the location that may have been sensitized to allergens. For example, by comparing the measurement results from these locations, the influence of baseline fluctuations in the body due to other diseases (for example, cold, hay fever) can be excluded to more appropriately evaluate the disease progression in the affected area (the location sensitized to allergens, and the location that may have been sensitized to allergens).

The comparison of the measurement results between the normal location and the location sensitized to allergens and the location that may have been sensitized to allergens, and the baseline correction may be performed, for example, in the determination unit 5, along with other operations.

When performed on a normal location that is not sensitized to allergens, the procedures including irradiation of terahertz light may be performed in one or more normal locations.

The effects described above become more prominent when procedures such as irradiation of terahertz light are performed on more than one normal location, and the disease progression in the affected area (the location sensitized to allergens, and the location that may have been sensitized to allergens) can be more appropriately evaluated.

In the configuration shown in the diagram, the drug application device 100 includes a battery 60 installed as a power supply source.

This can improve the operability of the drug application device 100.

While the preferred embodiment has been described above, the invention is not limited to the foregoing embodiment.

For example, the drug application device may have configurations different from the above.

For example, the drug application device may include a recording unit that records the information obtained from the reflected rays. This makes it possible to, for example, desirably manage changes in the extent of patient's the allergic symptoms over a time course. The drug application device also may include input means (including a means to read information such as barcode) for inputting patient information (for example, ID), so that the input information can be used to manage changes in the extent of allergic symptoms of a plurality of patients over a time course. The recording unit may be installed in the drug application device, or may be provided as an external storage medium (for example, such as an SD card).

The foregoing representative embodiment was described through the case where the drug application device includes the display unit. However, the drug application device is not required to include the display unit. The drug application device may be adapted to output signals (for example, electrical signals) for displaying information concerning cytokine in an externally provided display device.

The foregoing representative embodiment described the drug application device in which a terahertz light source is provided as the photoirradiation unit. However, the photoirradiation unit may irradiate terahertz light using light from an external light source. Specifically, the drug application device is not required to include a terahertz light source.

The foregoing representative embodiment described the drug supplying unit that is configured to push the drug out of the drug tube (drug container) with the roller (extrusion roller). However, the drug supplying unit is not limited to this configuration.

For example, the drug supplying unit may be adapted to apply the drug to skin by contacting an applicator such as a brush, a spatula, a stamp, or a pen-shaped object (for example, a felt or synthetic fiber chemical absorber configured to supply chemicals to skin).

The drug supplying unit also may be configured to, for example, jet or spray a liquid drug through a nozzle. In this way, a drug can be applied (imparted) to the target location with improved position selectivity.

When configured to jet (spray) a liquid drug through a nozzle, the drug supplying unit may spray a liquid drug in a mist under gas pressure (for example, air pressure), or may eject a drug in droplets according to the inkjet scheme. The drug supplying unit may spray a drug in a mist, for example, under the repulsive force of the positive or negative charge given to the drug. When jetting (spraying) a liquid drug through a nozzle, the drug supplying unit may eject the drug continuously or at specified time intervals, or may intermittently eject the drug every time the ejector travels a specified distance.

With these configurations, the applied drug quantity may be adjusted by adjusting, for example, the drug ejection amount per ejection, the number of times the drug is ejected, or the density of the droplets that contact the skin.

The foregoing embodiment described the drug supplying unit as having the tube holder. However, the drug supplying unit may be provided with a tank for storing the injected specific drug.

The foregoing embodiment described storing (holding) a single drug (drug container). However, it is also possible to store (hold) a plurality of drugs (drug containers) . More specifically, for example, it is possible to store a plurality of drugs with different active ingredients, or a plurality of drugs with different concentrations (contents) of the same active ingredient. In this way, a drug may be selected according to disease progression. A plurality of drugs may be used together in appropriate proportions according to disease progression or other disease conditions.

The drug application device of the embodiment of the invention may be adapted to separately store (hold) a drug with an active ingredient (at least one drug) and a base material.

In this way, for example, the drug and the base material can be used by being mixed in appropriate proportions according to conditions such as disease progression, and the concentration (content) of the active ingredient can be desirably adjusted.

When storing a plurality of drugs, or when separately storing one or more drugs and the base material, the determination unit, which determines a drug supply using the output information from the detector, can desirably determine the type of drugs applied to different locations of the skin, or the proportions of drugs, and the contents of the active ingredients of drugs when a plurality of drugs is applied in combination.

The foregoing representative embodiment described the drug application device in which the drug container holder is installed. However, a drug may be externally supplied to the drug application device.

The foregoing representative embodiment was described through the case where the check unit checks the drug type (the type of the drug container containing a specific amount of a specific drug) by the size of the drug container itself, or the position, the shape, or the size of the notch provided in the drug container. However, the check unit may check the drug type by the surface shape of the drug container (for example, the printing, marks, or barcode on the drug container (barcode may be one- or two-dimensional barcode or a matrix or composite QR code), or the color or indentation patterns on the container). When the drug container used has an IC chip or a check electrode, the check unit may represent a reading means that reads information indicative of the drug type (the type of the drug container containing a specific amount of a specific drug).

When the determination unit is adapted to, for example, determine the need to apply a drug, or the applied drug quantity on the basis of whether the output value from the detector exceeds the threshold value, the drug application device of the embodiment of the invention may include a switch for switching the threshold value for the determination unit according to the type of drug.

The drug application device of the embodiment of the invention may include an alarm unit for warning that, for example, the drug applied to skin is not enough, or the remaining drug is low. For example, the alarming unit may warn users either visually (for example, by images or a lamp) or by sound (for example, a buzzer, or voice guidance).

The foregoing representative embodiment was described through the case where the drug application device is a handy device that can be held with hand. However, the drug application device of the embodiment of the invention may be a larger device with, for example, a mount (stage) on which a patient can rest at least a part of his/her body. The device may then scan a predetermined region of the patient's skin resting on the mount, and apply a drug, as needed.

The foregoing embodiment described the configuration in which a battery is installed in the drug application device. However, the device may be configured so that power is supplied from an external power supply.

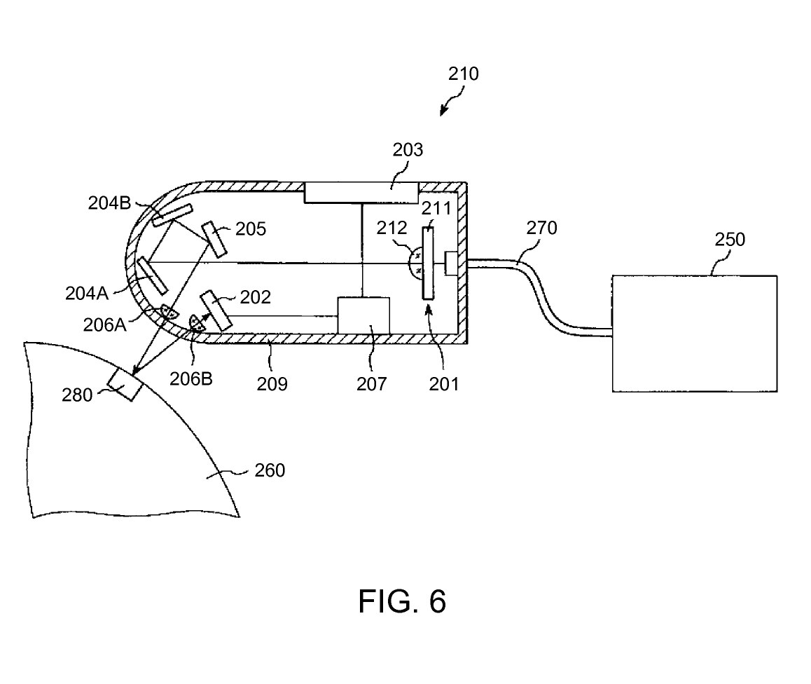

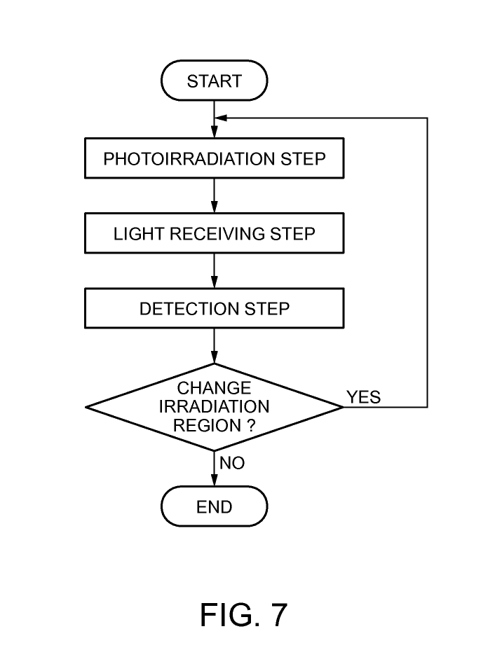

FIG. 6 is a partial cross sectional view schematically representing a preferred embodiment of a detection device according to the invention. FIG. 7 is a flowchart representing an example of a detection method according to the invention.

As represented in FIG. 6, a detection device 210 includes a photoirradiation unit 201 that irradiates terahertz light onto a patch 280 attached to skin 260, a light receiving unit 202 that receives reflected rays of the terahertz light, and an output unit 203 that outputs information concerning detection target cytokine determined from the reflected rays.

As represented in FIG. 7, the detection method of an embodiment of the invention includes a photoirradiation step of irradiating terahertz light onto a patch attached to skin, a light receiving step of receiving reflected rays of the terahertz light, and a detection step of obtaining information concerning detection target cytokine determined from the reflected rays.

It is known that cytokines (for example, such as TARC (thymus and activation-regulated chemokine)) are produced in locations sensitized to allergens in allergic diseases (for example, such as food allergy, allergic drug eruption, and contact dermatitis).

The terahertz light is light that easily passes through the patch 280, and that moderately passes through and reflects on skin, particularly the epidermis. A cytokine absorbs terahertz light in a specific absorption spectrum that varies with the chemical structure.

With the foregoing configuration, the cytokine produced in an allergic biological reaction in the skin (epidermis) can thus be desirably detected. The content of a predetermined cytokine in a location irradiated with terahertz light can be determined by finding the intensity of the reflected rays of the terahertz light of a predetermined wavelength (the proportion relative to the intensity of the irradiated light). This desirably enables not only detection of the presence or absence of predetermined cytokine production but quantitative analysis of the cytokine. The detection result can thus be desirably used not only in deciding whether a drug needs to be imparted or administered to the predetermined location but in determining the type or the quantity (dose) of the drug that needs to be imparted or administered. It is also possible to, for example, desirably decide the allergen quantity that needs to be given to a patient in an oral challenge therapy.

With the quick acquisition of information concerning cytokine, a treatment such as drug application can be quickly and appropriately performed. The extent of allergy also can be desirably determined with the patch attached in place, without the need to remove the patch to determine the extent of allergy. This enables monitoring the time-course changes as they occur in the location where the patch is attached. For example, there are cases where the extent of allergy cannot be easily determined after a predetermined time period from when the patch was attached. In this case, the extent of allergy can be desirably determined by irradiating the terahertz light again after a lapse of time with the patch, and there is no need to repeat the test with a new patch. Traditionally, a patch test requires about 48 hours with the patch before determination can be made. In contrast, the invention requires a shorter time period before the first determination, and the patch time (measurement time) can be extended, as required. Because the time required to make allergy determination can be greatly reduced as a whole, a burden on patients can be reduced, and an appropriate treatment can be quickly started.

The invention is also desirable for use in oral challenge therapy because the invention enables the sensitivity to specific allergens to be appropriately evaluated at different time points. To describe more specifically, oral challenge therapy is a regimen given to safely acquire resistance to allergens, whereby an allergen is given in increasingly larger doses that do not pose the risk of anaphylaxis. Allergen doses are specified by guidelines, and these doses do not take into account the symptoms and other conditions of a patient. For this reason, it is not unusual to see an allergic symptom developing during the oral challenge therapy, and forcing the patient to suspend the treatment. In contrast, the invention, by enabling the resistance to allergens to be appropriately evaluated at different time points, can more appropriately determine the allergen dose for oral challenge therapy, and effectively prevent the foregoing problems.

These desirable effects cannot be obtained with light other than terahertz light. For example, light having shorter wavelengths than terahertz light (for example, infrared rays) exposes the dermis through the epidermis, and the result is affected by dermis and blood vessels, which makes it difficult to accurately detect specific cytokines. With light having longer wavelengths than terahertz light (for example, milliwaves), the characteristic absorption spectrum of a cytokine will not be observed, and it is difficult to accurately detect specific cytokines.

The patch 280 may be configured from any material. Preferably, the patch 280 is configured from a material that contains at least one selected from the group consisting of polyethylene, polystyrene, polyvinyl chloride, and polyvinylidene chloride.

These materials have particularly low absorbance for terahertz light, and desirably allow cytokine detection in the location where the patch 280 is attached. Further, with their waterfastness and other desirable properties, the materials can more effectively prevent problems such as entry of outside moisture, and efflux of allergens due to moisture during a patch test.

For a patch test, the patch 280 typically contains an allergen. Examples of allergens include food allergens such as fruits (e.g., apple, avocado, banana, melon, cherry, coconut, red grape, grapefruit, kiwi, lemon, mango, orange, papaya, peach, pineapple, strawberry, and watermelon), nuts and cereals (e.g., almond, red bean, brown rice, cashew nut, soba, corn, wheat (such as whole-grain wheat, and wheat gluten), kidney bean, green gram (mung bean), oat, peanut, pistachio, rice, rye, sesame, soybean, walnut, and green bean), dairy products (e.g., casein, cheddar cheese, cottage cheese, milk, whey (milk serum), and yogurt), meat (e.g., cow, chicken, chicken egg (egg yolk), chicken egg (egg white), lamb, pig, abalone, hamaguri, cod, crab, squid, oyster, red snapper, salmon, perch, shrimp, tuna, scallop and shellfish), vegetables (e.g., bamboo shoots, bean sprout, bitter gourd, broccoli, cabbage, carrot, cauliflower, celery, cucumber, eggplant, garlic, green pepper, kelp, leek, lettuce, mushroom, olive, onion, squash, spinach, sweet potato, tomato, and potato), spices (e.g., curry powder, ginger, mustard, black pepper, chili, and vanilla), eggs (e.g., ovomucoid, and egg white), bread yeast, brewery yeast, cacao, coffee, honey, sugar cane, and green tea; pollen allergens such as Timothy-grass, buffalo grass, cocksfoot, blackweed, Japanese mugwort, sugi (Japanese cedar), hinoki (Japanese cypress), hannoki (Japanese alder), and silver birch; environmental allergens such as Dermatophagoides farinae, house dust, cat dander, and dog dander; metal allergens (such as nickel, and cobalt), Penicillium, Cladosporium, Candida, Alternaria, Aspergillus, and latex.

The patch 280 may contain more than one allergen.

In this way, allergens producing an allergic response can be efficiency specified.

When the patch 280 contains more than one allergen, the allergens may be contained in different regions, or may be contained as a mixture.

When the patch 280 contains an allergen, the patch 280 contains an allergen over an area of preferably 0.01 cm² to 0.25 cm², more preferably 0.02 cm² to 0.20 cm².

By using terahertz light for detection, the invention enables a more accurate evaluation of an allergic response in skin, despite that the patch is smaller than patches used in common patch tests (patch tests involving evaluation by visual inspection) . A burden on patients also can be reduced with the small patch (a patch with a small contact area between the skin and allergens). The small patch also has other advantages, including shipping cost, storage space, and resource saving.

The patch 280 has a moisture content of preferably 0.5 g/cm² or less, more preferably 0.2 g/cm² or less per unit area in a region irradiated with the terahertz light.

This further improves the cytokine detection accuracy, and enables a more desirable evaluation of an allergic response in skin.

The cytokine may be, for example, any of various interleukins (IL) such as IL-5, 6, 8, 13, and 33; TSLP (thymic stromal lymphopoietin); hematopoietic factors such as GM-CSF (granulocyte macrophage colony stimulating factor); and various chemokines, including CC chemokines such as CCL 2, 22 (MDC), and 24 (Extaxin-2), CXC chemokines such as CXCL 1 and 8, C chemokines, and CX3C chemokines.

Terahertz light has a wavelength of 0.5 THz to 10 THz. However, the wavelength of the terahertz light irradiated by the photoirradiation unit 201 ranges preferably from 0.8 THz to 3.0 THz, more preferably 1.0 THz to 2.0 THz.

In this way, various cytokines can be quantitatively detected with improved accuracy.

The detection device 210 may include a terahertz light-emitting light source as a part of its configuration, or may be configured so that the photoirradiation unit 201 irradiates light from an external light source 250 guided through an optical fiber 270.

This makes it possible to miniaturize the detection device 210. It also becomes possible to effectively make use of an existing light source.

In the present embodiment, the photoirradiation unit 201 is optically connected to the external light source 250. The light (near-infrared rays) emitted from the femtosecond laser light source provided as the external light source 250 is subjected to wavelength conversion by a photoconductive antenna 211, and terahertz light is radially generated. The terahertz light is then emitted as parallel rays through a lens 212.

In this way, terahertz light can be desirably irradiated with a device (light source) that is readily available at relatively low cost.

The terahertz light emitted by the photoirradiation unit 201 is reflected by fixed mirrors 204A and 204B and a movable mirror 205, and irradiates the patch 280 attached to skin 160 after being converted into parallel rays through a lens 206A. The provision of the different mirrors makes it possible to more freely dispose the components of the detection device 210, and miniaturize the detection device 210. The movable mirror 205 enables the terahertz light to scan a predetermined region of the patch 280.

The reflected rays of the skin 260 in the tested location (a portion of the skin 260 covered with the patch 280) converge into the light receiving unit 202 through a lens 206B.

The light receiving unit 202 is configured from an optical sensor that can detect terahertz light.

The optical sensor used to form the light receiving unit 202 may be, for example, a thermal sensor or a quantum sensor, specifically a photodiode. The output current from the photodiode increases as the quantity of the detected light increases. The output from the light receiving unit 202 is sent to a signal processing unit 207.

The light receiving unit 202 may include a single sensor, or may include a plurality of sensors that is arranged in lines on a chip or arrayed on a surface of a chip.

The light receiving unit 202 may include a bandpass filter (for example, a filter that selectively passes light of a predetermined wavelength in the wavelength region of the terahertz light).

This further improves the detection accuracy of predetermined cytokine. The need for a frequency sweep also can be eliminated to simplify the device configuration.

When the patch 280 contains a substance such as an allergen, the bandpass filter can effectively prevent, for example, the existence of such a substance from adversely affecting the cytokine detection.

Preferably, the bandpass filter selectively passes light of the wavelength corresponding to the absorption peak of the absorption spectrum of the terahertz light for a specific cytokine.

This further improves the detection accuracy of predetermined cytokine.

The accuracy of specifying different cytokines can improve when the bandpass filter is adapted to selectively pass light of different wavelengths corresponding to the characteristic absorption peaks (different absorption peaks) of different cytokines.

The output from the light receiving unit 202 is arithmetically processed in the signal processing unit 207. Information concerning detection target cytokine (for example, the presence or absence of production of a specific cytokine, and the amount or the type of the cytokine contained in the location where the patch 280 is attached) can then be obtained from the quantity (intensity) of the reflected terahertz light of a specific wavelength, using, for example, data of a separately prepared standard curve.

The optical members, including the photoirradiation unit 201 and the light receiving unit (sensor) 202, and the signal processing unit 207 are housed inside a casing (housing) 209, and are desirably protected.

The casing 209 also serves as a holder when operating the detection device 210.

The output unit 203 outputs information concerning detection target cytokine determined from the reflected rays.

In the present embodiment, the output unit 203 is a display unit that displays information concerning cytokine.

The display unit allows users (e.g., patients) of the detection device 210 to, for example, easily recognize information concerning cytokine. The display unit also eliminates the need to connect the detection device 210 to an external device, for example, such as a display device for displaying the output of the output unit 203, and a recording device that records the output of the output unit 203. This allows users to recognize information concerning cytokine with a simple configuration.

The output unit (display unit) 203 may be provided at any location. In the present embodiment, the output unit (display unit) 203 is provided in the casing (housing) 209.

In this way, information concerning cytokine can be easily recognized while effectively preventing the size of the detection device 210 from increasing.

The output unit 203 is not particularly limited, as long as it outputs information concerning detection target cytokine determined from the reflected rays. The information maybe any of, for example, the content or the type of cytokine, and the type or the quantity of the drug to be imparted as determined from the cytokine information.

Preferably, the output unit 203 outputs information concerning a content of TARC (thymus and activation-regulated chemokine).

Compared to other cytokines, TARC is more prominently produced in locations sensitized to allergens. TARC also has an absorption spectrum that can be more easily distinguished from other substances with terahertz light. With the output unit 203 outputting information concerning a TARC content, the extent of allergic symptoms can thus be more appropriately determined, and, for example, conditions such as the type and the quantity of the drug to be imparted can be more desirably determined. It is also possible to, for example, desirably determine the allergen dose to be given to a patient in an oral challenge therapy.

Examples of the information concerning a TARC content include the content of TARC in a location irradiated with terahertz light (including the presence or absence of TARC), the type or the quantity (dose) of the drug to be imparted or administered as determined from the content, and the allergen dose to be given in an oral challenge therapy.

The output unit (display unit) 203 may display information concerning cytokine in characters (e.g., numerical values) or images. For example, a distribution of cytokine contents at different locations of the region irradiated with terahertz light may be displayed in colors.

This makes it easier to visually determine the extent of allergic symptoms.

The foregoing procedures including the irradiation and the subsequent reception of terahertz light are intended for the patch 280 attached to the skin 260. However, the procedures including the irradiation of terahertz light also may be performed in locations of the skin 260 where the patch 280 is not attached, in addition to the patch 280 attached to the skin 260.

In this way, by using the location of the skin 260 with no patch 280 (normal location) as a reference location, the measurement result from the reference location can be compared with the measurement results from the location where the patch 280 is attached. For example, by comparing the measurement results from these locations, the influence of baseline fluctuations in the body due to other diseases (for example, cold) can be excluded to more appropriately evaluate the allergic response (e.g., the presence or absence of production of a predetermined cytokine, and the cytokine level) in the location where the patch 280 is attached.

The comparison of the measurement results between the reference location and the location where the patch 280 is attached, and the baseline correction may be performed, for example, in the signal processing unit 207, along with other operations.

When procedures such as irradiation of terahertz light are performed on a reference location, these procedures may be performed in one or more reference locations.

The effects described above become more prominent when procedures such as irradiation of terahertz light are performed on more than one reference location, and the allergic response (e.g., the presence or absence of production of a predetermined cytokine, and the cytokine level) in the location where the patch 280 is attached can be more appropriately evaluated.

While the preferred embodiment has been described above, the invention is not limited to the foregoing embodiment.

For example, the detection device may have configurations different from the above.

For example, the detection device may include a recording unit that records the information obtained from the reflected rays. This makes it possible to, for example, desirably manage changes in the extent of the patient's allergic symptoms over a time course. The detection device also may include input means (including a means to read information such as barcode) for inputting patient information (for example, ID), so that the input information can be used to manage changes in the extent of allergic symptoms of a plurality of patients over a time course. The recording unit may be installed in the detection device, or may be provided as an external storage medium (for example, such as an SD card).

The foregoing representative embodiment was described through the case where the detection device includes the display unit as the output unit. However, the output unit provided for the detection device is not limited to this embodiment, as long as the output device outputs information concerning cytokine. For example, the output device may be adapted to output signals (for example, electrical signals) for displaying information concerning cytokine in an externally provided display device.

The foregoing representative embodiment was described through the case where the photoirradiation unit irradiates terahertz light using light from an external light source. However, the detection device may be adapted to include a terahertz light source.

The foregoing embodiment was described through the case where the patch basically contains an allergen. However, the invention may use a patch that does not contain an allergen. For example, the invention may be applied to determine when to remove or replace a patch by detecting and evaluating the extent of symptoms of a patient with an allergic skin disease in a location showing symptoms such as inflammation after imparting a drug and protecting the location with a patch (including attaching a patch containing a drug) using the terahertz light.

The entire disclosure of Japanese Patent Application No. 2015-117659 filed Jun. 10, 2015 and No. 2015-117658 filed Jun. 10, 2015 are expressly incorporated by reference herein.

What is claimed is:

1. A drug application device comprising:
a photoirradiation unit that irradiates terahertz light onto skin;
a light receiving unit that receives reflected rays of the terahertz light;
a detector that outputs information concerning cytokine detected from the reflected rays;
a drug supplying unit that supplies a drug to the skin; and
a determination unit that determines a supply of the drug on the basis of the output information from the detector.

2. The drug application device according to claim 1, comprising a display unit that displays the information concerning cytokine.

3. The drug application device according to claim 1, comprising a determination unit that determines a supply of the drug on the basis of whether the output value from the detector exceeds the threshold value.

4. The drug application device according to claim 1, wherein the drug supplying unit adjusts a supplied drug quantity according to the information from the detector.

5. The drug application device according to claim 1, comprising a check unit that checks a type of the drug.

6. The drug application device according to claim 1, comprising an application roller that rolls on the skin and applies the drug to the skin.

7. The drug application device according to claim 1, comprising an extrusion roller that presses a container storing the drug and extrudes the drug out of the container.

8. The drug application device according to claim 1, comprising a drug container holder that holds a drug container containing the drug.

9. The drug application device according to claim 1, wherein the detector outputs information concerning a content of TARC (thymus and activation-regulated chemokine).

10. A detection device comprising:
a photoirradiation unit that irradiates terahertz light;
a light receiving unit that receives reflected rays of the terahertz light;
a detector that outputs information concerning cytokine detected from the reflected rays; and
a display unit that displays the information concerning cytokine.

11. The detection device according to claim 10, wherein the terahertz light irradiates a patch attached to skin.

12. The detection device according to claim 11, wherein the patch has a moisture content of 0.5 g/cm² or less per unit area in a region irradiated with the terahertz light.

13. The detection device according to claim 11, wherein the patch is configured from a material that contains at least one selected from the group consisting of polyethylene, polystyrene, polyvinyl chloride, and polyvinylidene chloride.

14. The detection device according to claim 11, wherein the patch contains an allergen, and an area of a part of the patch where the allergen is contained is 0.01 cm² to 0.25 cm².

15. The detection device according to claim 10, wherein the detector outputs information concerning a content of TARC (thymus and activation-regulated chemokine).

16. The detection device according to claim 10, wherein the terahertz light is produced with a photoconductive antenna by wavelength conversion from light emitted by a femtosecond laser light source.

17. A drug application method comprising:
irradiating terahertz light onto skin;
receiving reflected rays of the terahertz light;
outputting information concerning detection target cytokine determined from the reflected rays; and
supplying a drug to the skin on the basis of the information concerning cytokine.

18. A detection method comprising:
irradiating terahertz light onto a patch attached to skin;
receiving reflected rays of the terahertz light; and
obtaining information concerning detection target cytokine determined from the reflected rays.
